(19) United States
(12) Patent Application Publication
(10) Pub. No.: US 2025/0369838 A1
(43) Pub. Date: Dec. 4, 2025

(54) LIPOASPIRATE CELLULARITY AND MECHANICAL PROCESSING METHODS

(71) Applicant: Greyledge Technologies, LLC, Avon, CO (US)

(72) Inventors: David Karli, Eagle, CO (US); Theodore Sand, Austin, TX (US); Mark Kitchen, San Antonio, TX (US); Robert Jones, Lago Vista, TX (US)

(73) Assignee: Greyledge Technologies, LLC, Avon, CO (US)

(21) Appl. No.: 18/872,831

(22) PCT Filed: Jun. 8, 2023
(86) PCT No.: PCT/US2023/024813

§ 371 Date: Dec. 8, 2024

Related U.S. Application Data
(60) Provisional application No. 63/350,394, filed on Jun. 8, 2022.

(51) Int. Cl. G01N 1/28; G01N 1/40

(52) U.S. Cl. CPC G01N 1/286; G01N 1/4044

(57) ABSTRACT

A system and method are disclosed that allows for the standardization of the application of physical force during microfragmentation of adipose tissue with enhanced sterility. A method is also disclosed that enables an accelerated digestion of a portion of the microfragmented adipose tissue that allows for at least an estimation of the viability of the released cells, as well as a particle/cell count. A further method is disclosed to provide for assessing attributes of the microfragmented adipose tissue, including physical, biochemical and metabolic characteristics of particles/cells present in and/or isolated from the microfragmented adipose tissue.

### CROSS-REFERENCE TO RELATED APPLICATIONS

This patent application claims priority from, and incorporates by reference the entire disclosure of, U.S. Provisional Patent Application No. 63/350,394 filed on Jun. 8, 2022.

### TECHNICAL FIELD

The present disclosure relates generally to regenerative treatments and more particularly, but not by way of limitation, to fragmentation of adipose tissue for use in regenerative treatments.

### BACKGROUND

This section provides background information to facilitate a better understanding of the various aspects of the disclosure. It should be understood that the statements in this section of this document are to be read in this light, and not as admissions of prior art.

One common method for utilizing a patient's own cells for regenerative treatments is to mechanically fragment adipose tissue, and subsequently process the fragmented adipose tissue for implantation into the patient. Mechanical fragmentation of adipose tissue can be achieved with a variety of commercially available kits. Mechanical fragmentation of adipose tissue can be achieved by manual methods involving the application of physical force to reduce the size of adipose tissue particles obtained from a lipoaspiration procedure. Mechanical fragmentation, also known as microfragmentation or micronization, results in adipose tissue particles that have a reduced number of intact adipocytes enmeshed in connective tissue. The microfragmented tissue contains a wide variety of viable cells, including fibroblasts, white blood cells, red blood cells, platelets, endothelial cells, mesenchymal stromal cells, etc.

### SUMMARY OF THE INVENTION

This summary is provided to introduce a selection of concepts that are further described below in the Detailed Description. This summary is not intended to identify key or essential features of the claimed subject matter, nor is it to be used as an aid in limiting the scope of the claimed subject matter.

A system and method are disclosed that allows for the standardization of the application of physical force during microfragmentation of adipose tissue with enhanced sterility. A method is also disclosed that enables an accelerated digestion of a portion of the microfragmented adipose tissue that allows for at least an estimation of the viability of the released particles that are nucleated, as well as a particle count. A further method is disclosed to provide for assessing attributes of the microfragmented adipose tissue, including the biochemical and metabolic characteristics of cells present in and/or isolated from the microfragmented adipose tissue.

### BRIEF DESCRIPTION OF THE DRAWINGS

A more complete understanding of the subject matter of the present disclosure may be obtained by reference to the following Detailed Description when taken in conjunction with the accompanying Drawings wherein:

FIG. 1 illustrates a processing system according to aspects of the disclosure;

FIG. 2 illustrates a mechanical processing device according to aspects of the disclosure;

FIG. 3A illustrates a mechanical processing device according to aspects of the disclosure;

FIG. 3B is a partial exploded assembly of the mechanical processing device of FIG. 3A according to aspects of the disclosure;

FIG. 3C is a sectioned view of the mechanical processing device of FIG. 3A according to aspects of the disclosure;

FIG. 3D is a detail view of the mechanical processing device of FIG. 3C;

FIG. 4 illustrates a spring-loaded return device according to aspects of the disclosure;

FIG. 5 is a close-view of a retention mechanism of the spring-loaded return device according to aspects of the disclosure; and

FIGS. 6A and 6B illustrate spring-loaded return device in use according to aspects of the disclosure.

### DETAILED DESCRIPTION

It is to be understood that the following disclosure provides many different embodiments, or examples, for implementing different features of various embodiments. Specific examples of components and arrangements are described below to simplify the disclosure. These are, of course, merely examples and are not intended to be limiting. The section headings used herein are for organizational purposes and are not to be construed as limiting the subject matter described.

Some commercially available kits use a connector of fixed diameter to reduce the particle size of lipoaspirated adipose tissue. The adipose tissue is forced through the connector via a user's actuation of a plunger of a syringe. As the adipose tissues passes through the connector, the size of the adipose tissue particles is reduced. Kits such as these suffer from sterile breaks (e.g., seven or more sterile breaks) that occur when the two syringes are connected to the connectors, unless the processing occurs in a sterile field. There is also the issue of variable force and flow rate, since the movement of the tissue relies on a user manually forcing the plunger of the syringes.

There is no standard method or kit for analyzing the cellular compositions of microfragmented adipose tissue. Two approaches have been published: 1) digest the adipose tissue and analyze the released cells with a variety of analytical methods (e.g., cluster of differentiation markers) immediately after isolation or after being placed in tissue culture; or 2) histochemical and immunohistochemical methods of characterizing the cells in the adipose tissue without isolation, including light microscopy and confocal immunofluorescence microscopy. The microfragmented adipose tissue pieces also have been placed into explant culture in order to estimate the number of cells present in the tissue explants over several weeks.

The inventive device and system of the instant application standardizes the amount of force used to fragment adipose tissue, which is in contrast with the currently commercially available kits, methods and devices that all require manual manipulation by an operator and hence are inherently variable. The inventive system will be placed in a Biological Safety Cabinet Class II Type 2A (BSC), which virtually eliminates the threat of contamination by adventitious agents, since the interior of the BSC is rated as ISO 5 (i.e., sterile) and is used without the need to have a sterile field established in an operating room. Unless the existing commercial devices are only used in the sterile field of an operating room, there are multiple sterile breaks in the processing of adipose tissue with those kits and devices, which might lead to an infection in the patient. While it is possible for a physician to perform the manipulations with a commercially available kit in a BSC of a clinic, a BSC isn't routinely found in clinics. Furthermore, some automated processing devices still require connections of saline and introduction of the lipoaspirated adipose tissue, as well as recovery of the microfragmented adipose tissue after processing, which, unless performed in a sterile field, constitute sterile breaks. In contrast, there are no sterile breaks associated with the inventive device. In addition to eliminating sterile breaks, the inventive method for characterizing the particles, including nucleated cells, of the microfragmented adipose tissue will provide a near-real time estimate of the number of particles and nucleated cells, their viability, and other cellular attributes (i.e., “cellularity”).

Reference will now be made to more specific embodiments of the present disclosure and data that provides support for such embodiments. However, it should be noted that the disclosure below is for illustrative purposes only and is not intended to limit the scope of the claimed subject matter in any way.

A system and device are described that facilitates the microfragmentation of lipoaspirated adipose tissue under sterile and controlled conditions. A portion of the micronized adipose sample is immediately transferred into a separate portion of the system and device and therein the particles and nucleated cells are released from the micronized adipose tissue by the inventive method to characterize the particles and nucleated cells present in the micronized adipose tissue preparation, as well as to characterize the particles and nucleated cells present in the unfragmented lipoaspirated adipose tissue (the “raw” sample). The system and method for microfragmenting the adipose tissue and the system and method for characterizing the raw and micronized adipose tissue are documented, and details of the processing of each sample is recorded contemporaneously.

### Mechanical Processing of Lipoaspirate

The processing of a lipoaspirate sample is performed in a BSC. Prior to initiating the mechanical process, syringes and connectors will be placed in the BSC. A syringe pump that is able to infuse and withdraw and can accommodate an appropriately-sized syringe (e.g., 20, 30 or 60 mL) is used to provide mechanical force. The syringe pump should be able to provide sufficient force to move lipoaspirated adipose tissue at rates of 1 mL/min up to 100 mL/min or more through defined diameter conduits.

The lipoaspirated adipose tissue is obtained by a physician and transferred to the BSC for mechanical processing. If necessary, the syringe(s) is placed in a vertical position to allow any tumescent fluid or blood to collect at the bottom of the syringe or free lipid at the top. All handling of syringes with a patient's lipoaspirated adipose tissue will occur within the sterile working area of the BSC. Prior to transferring the adipose tissue sample to a primary syringe, any fluid that has pooled at the top of the syringe barrel is expelled. The condensed adipose tissue is transferred to the primary syringe.

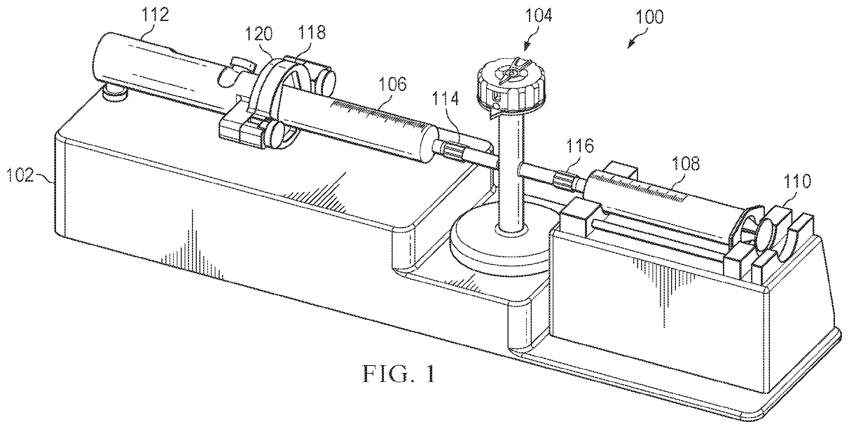

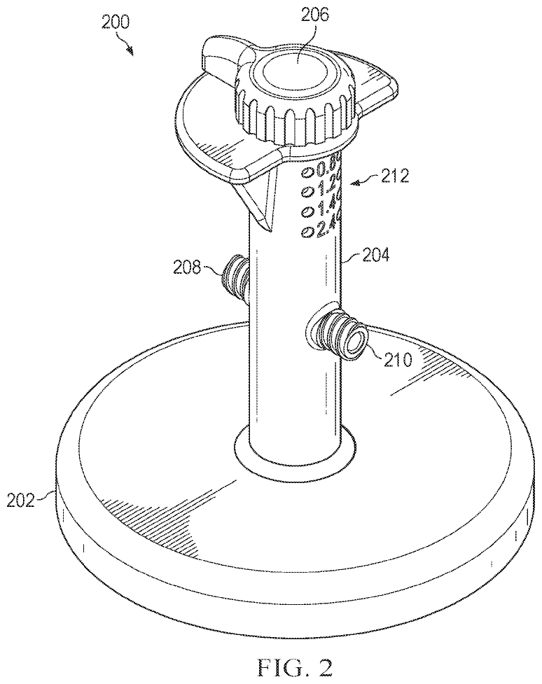

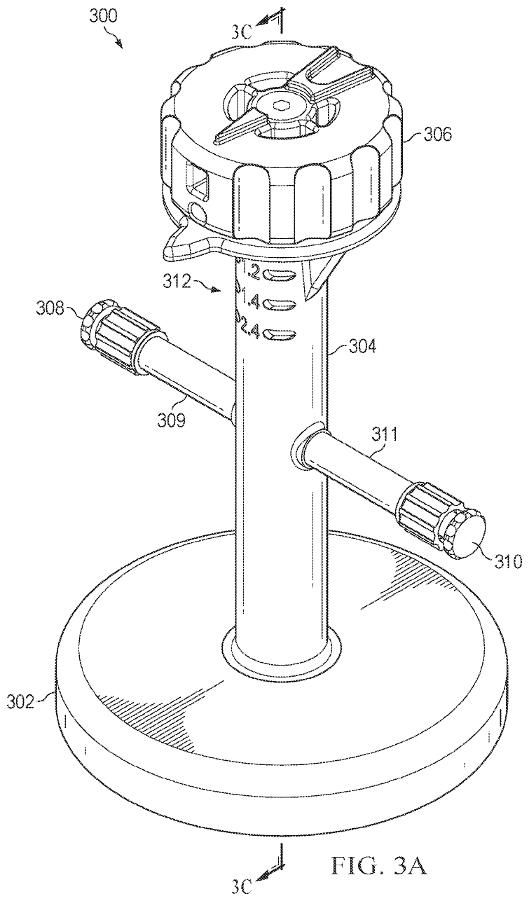

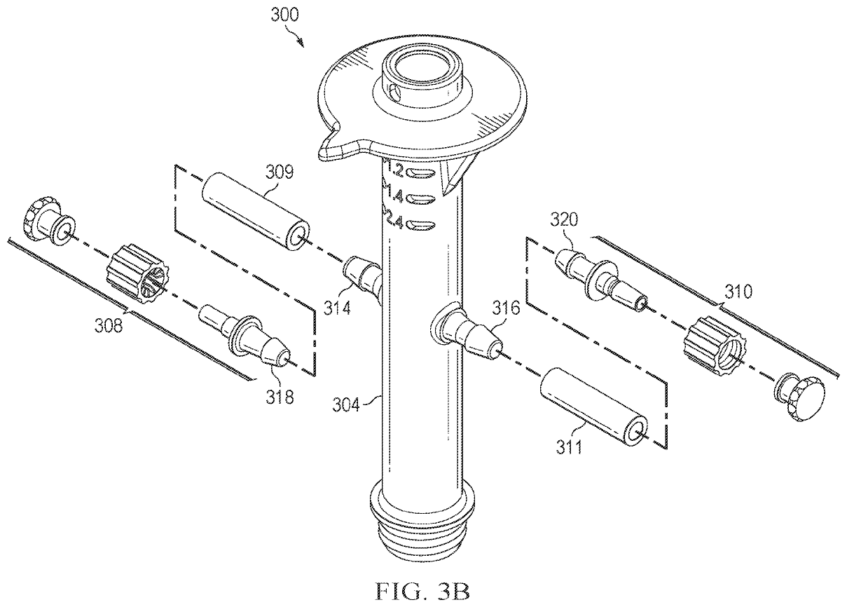

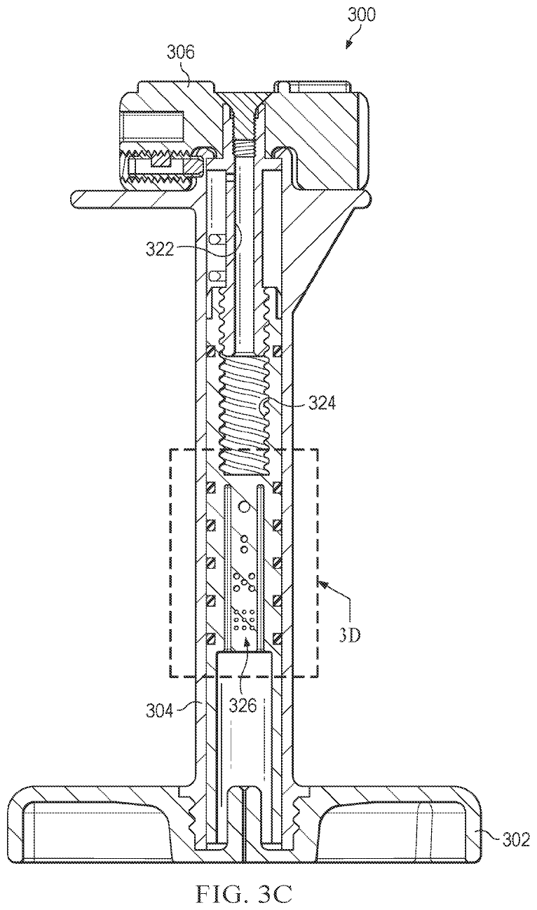

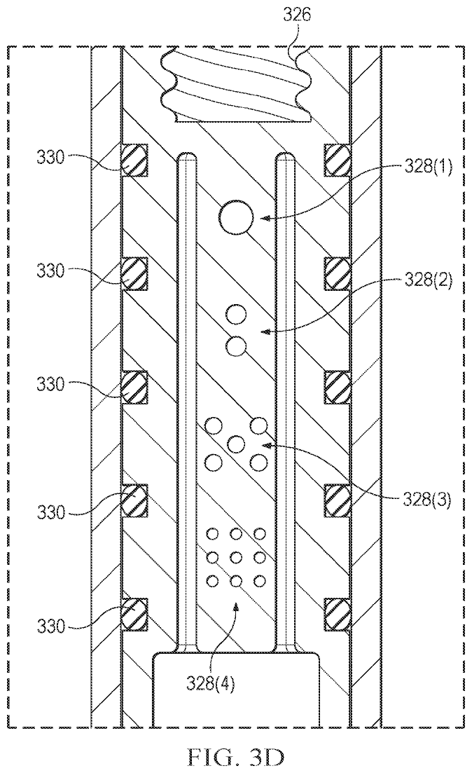

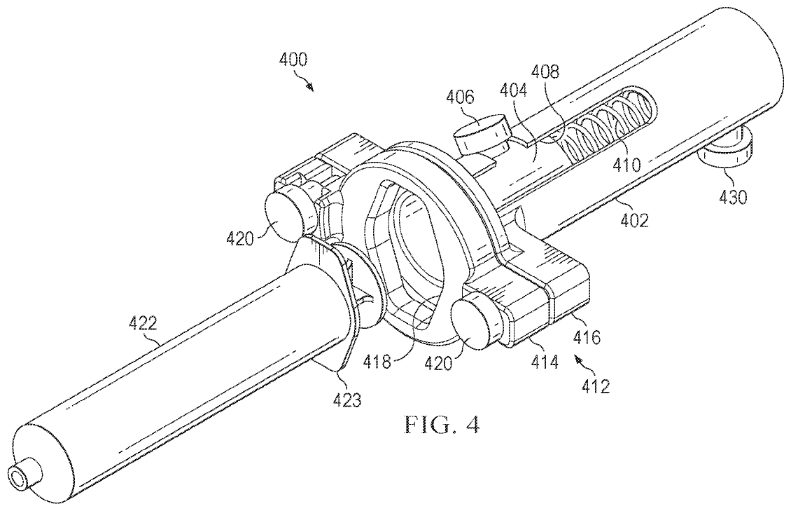

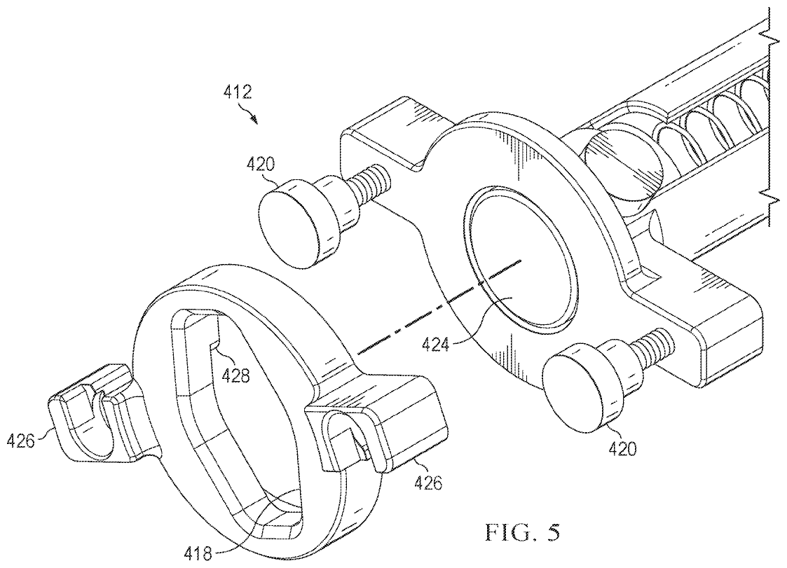

FIG. 1 illustrates a processing system 100 that includes a base 102. Base 102 is configured to receive a Mechanical Processing Device (“MPD”) 104 that couples a receiving syringe 106 to a primary syringe 108. MPD 104 may be secured to housing 102. Housing 102 includes a cradle 110 in which primary syringe 108 is secured. Embodiments of an MPD are discussed in more detail relative to FIGS. 2 and 3A-3D. System 100 also includes a spring-loaded return device 112 that is configured to manipulate receiving syringe 106 to expel fluids therefrom. Embodiments of spring-loaded return devices are discussed in more detail relative to FIGS. 4, 5, and 6A-6B. During operation, MPD 104 is coupled between receiving syringe 106 and primary syringe 108 via ports 114, 116, respectively. A flanged end 118 (i.e., a barrel flange) of receiving syringe 106 is coupled to a retention mechanism 120 of spring-loaded return device 112. Securing receiving syringe 106 is discussed in more detail relative to FIG. 5.

FIG. 2 illustrates a configuration of an MPD 200 according to aspects of the disclosure. MPD 200 may be used with processing system 100 and may take the place of MPD 104 of FIG. 1. MPD 200 includes a base 202, a housing 204, and an adjustment control 206. Housing 204 includes Luer-Lok fittings 208, 210 that are configured to couple to receiving syringe 106 and primary syringe 108, respectively. Adjustment control 206 allows a user to select a conduit size for fluid passing through MPD 200. Adjustment control 206 is connected to a shaft that extends into housing 204. The shaft includes a plurality of apertures of different sizes/numbers through which fluid passes during processing. Turning adjustment control 206 axially moves the shaft to align the plurality of apertures with the flow path between receiving syringe 106 and primary syringe 108 (see discussion relative to FIGS. 3A-3D below). Housing 204 includes a plurality of visual indicator windows 212 that provide a visual indication of the alignment of the shaft within housing 204 so that a user can visibly see which processing size has been selected.

FIGS. 3A-3D illustrate an MPD 300 according to aspects of the disclosure. MPD 300 is similar to MPD 200 and may be used in place of MPD 104 of FIG. 1. MPD 300 includes a base 302, a housing 304, and an adjustment control 306. MPD 300 includes Luer-Lok fittings 308, 310 that are connected to housing 304 via flexible tubing 309, 311, respectively. Flexible tubing 309, 311 can make connecting syringes 106, 108 as they allow for some movement of the Luer-Lok fittings 308, 310. FIG. 3B is an exploded assembly of MPD 300 with base 302 and adjustment control 306 removed. Housing 304 includes a pair of barbs 314, 316 that receive a first end of flexible tubing 309, 311, respectively. Luer-Lok fittings 308, 310 include barbs 318, 320 that similarly receive a second end of flexible tubing 309, 311.

FIG. 3C is a section view about A-A of FIG. 3A and FIG. 3D is a detail view of B of FIG. 3C. MPD 300 includes a shaft 322 that is coupled to adjustment control 306. Shaft 322 is coupled to a threaded body 324 that threadably engages conduit member 326. Turning adjustment control 306 axially moves conduit member 326 to align one of a plurality of conduits 328(1)-(4) with the flow path between receiving syringe 106 and primary syringe 108. Each conduit extends through conduit member 326. As shown in FIG. 3D, conduit 328(1) is a single conduit with a diameter of 2.4 mm, conduit 328(2) includes two conduits with a diameter of 1.4 mm each, conduit 328(3) includes five conduits with a diameter of 1.2 mm each, and conduit 328(4) includes nine conduits with a diameter of 0.8 mm each. In various embodiments, more or fewer conduits may be incorporated into conduit member 326. In various embodiments, the number and size of the conduits may be changed. In general, increasing the number of conduits while decreasing the size of the conduits allows for a reduction in the resistance of the flow through each conduit 328(1)-(4).

Visual indicator windows 312 on housing 304 provide a visual indication to the user as to which conduit 328(1)-(4) is aligned with the flow path. The windows are ports formed through housing 304 that allow the user to see a portion of conduit member 326 to provide an indication of which conduit 328(1)-(4) is currently aligned with the flow path. A plurality of seals 330 (e.g., o-rings) are arranged about conduit member 326 to seal the flow path. As shown in FIG. 3D, pairs of seals 330 are positioned vertically above and below a conduit 328 create a sealed flow path that channels fluid through a selected conduit 328.

FIG. 4 illustrates a spring-loaded return device 400 according to aspects of the disclosure. Device 400 may be used, for example, with processing system 100. Device 400 includes a housing 402 that has a plug 404 and a spring 410 disposed therein. Plug 404 acts as a piston that bears against an end of a syringe 422. Spring 410 biases plug 404 toward a return mechanism 414. Plug 404 includes a lockout 406 that extends through a slot 408 of housing 402. As shown in FIG. 4, lockout 406 is in a lockout position (i.e., engaged with a offshoot of slot 408). In the lockout position, plug 404 cannot translate axially and is displaced axially to slightly compress spring 410 to make it easier to couple syringe 422 to return mechanism 412 (i.e., plug 404 is retracted so as to not contact the end of syringe 422). A user may adjust the position of lockout 406 to disengage the lockout position to allow plug 404 to move axially within housing 402.

FIG. 5 is a close-up view of return mechanism 412. Return mechanism 412 includes cap 414 and a head 416. Cap 414 includes an opening 418 that is configured to receive a flange 423 of syringe 422. Opening 418 oblong in shape and follows the contours of flange 423, but is sized larger so that flange 423 may pass therethrough. Cap 414 is secured to head 416 via a pair of screws 420 that engage ears 426. Opening 418 is configured with a recessed edge 428 that is offset axially relative to a back face of head 416. Recessed edge 428 allows flange 423 to engage cap 414 to retain syringe 422 (e.g., receiving syringe 106). For example, flange 423 is inserted into opening 418 and rotated so that flange 423 extends over recessed edge 428 (e.g., rotated about 10-90 degrees). To remove syringe 422 from retention mechanism 412, flange 423 is rotated in the opposite direction. Once syringe 422 is engaged between cap 414 and head 416, a front surface 424 of plug 404 contacts the plunger of syringe 422 by moving lockout 406 out of the offshoot of slot 408.

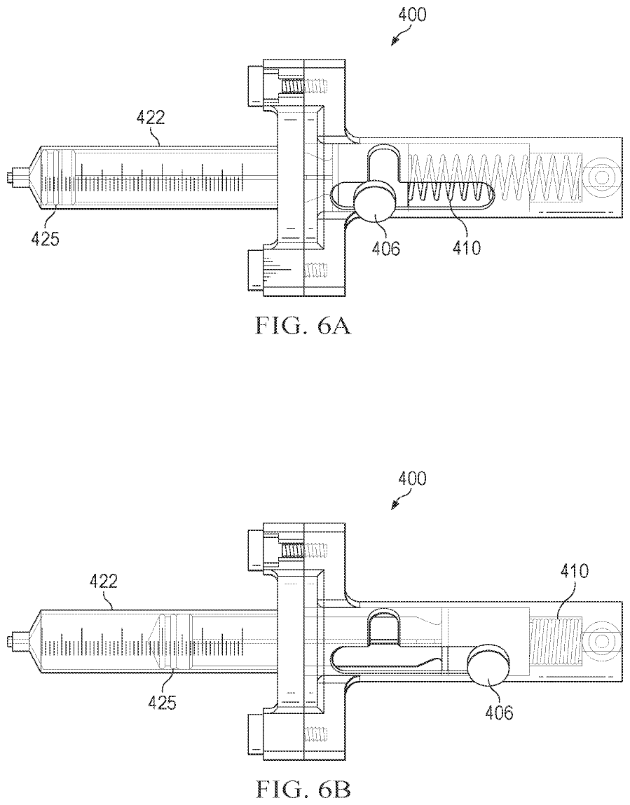

FIGS. 6A and 6B illustrate spring-loaded return device 400 in operation. Referring collectively to FIGS. 1, 4, 5, and 6A-6B, syringe 422 is coupled to return mechanism 412, with flange 423 engaged with recessed edge 428. During use, spring-loaded return device 400 rests on a foot 430 to maintain a horizontal alignment with primary syringe 108 that is seated in processing system 100. Spring-loaded return device 400 is arranged such that as the plunger of syringe 422 (e.g., receiving syringe 106) is displaced upon initiation of the “infuse” stroke performed by the syringe pump, the head of the plunger of syringe 422 moves against plug 404, compressing spring 410 against housing 402. At initiation of a processing cycle, the syringe pump pushes an adipose tissue sample (though other samples may be similarly processed) out of primary syringe 108, through a conduit (e.g., conduit 328(1)) of the MPD and into syringe 422. As syringe 422 fills with the processed adipose tissue, the plunger of syringe 422 pushes against plug 404, thereby compressing spring 410. Once all of the adipose tissue sample has been forced through the conduit into syringe 422, the “withdraw” portion of the cycle is initiated. Compressed spring 410 aids in the return transfer of the sample from syringe 422 through conduit 328 and into a primary syringe (e.g., primary syringe 108). Each cycle of mechanical fragmentation exerts the same amount of force, since the force is provided by the syringe pump. Spring 410 provides additional force to syringe 422 while the syringe pump withdraws the sample, which reduces the occurrence of cavitation. Cavitation can occur when a vacuum develops as the seal of the primary syringe is moving during the withdraw stroke, but the tissue mass and receiving syringe seal fail to move back in sync with the movement of the seal of the primary syringe.

The diameters of conduits 328 used during the microfragmentation of the sample can vary. For example, the initial processing can occur with a 2.4 mm diameter conduit and the sample can be cycled from 1, 2, 3, 4, 5, 10, 15, 20, 25, 30 or more times depending on the stiffness of the sample. After completing the target number of cycles with the first conduit 328, a second, smaller diameter conduit 328 (e.g., 1.4 mm) may be used. During this second stage of processing, an additional 1, 2, 3, 4, 5, 10, 15, 20, 25, 30 or more cycles will be performed. At the completion of the target number of cycles with the second conduit 328, a third conduit 328 with an even smaller diameter (e.g., 1.2 mm) may be used. During this third stage of processing, an additional 1, 2, 3, 4, 5, 10, 15, 20, 25, 30 or more cycles will be performed. Depending on the intended use, a three-stage processing sequence with 2.4 mm, 1.4 mm and 1.2 mm diameter lumen connectors will result in a suitable micronized sample preparation. It will be appreciated that conduit diameters used, the number of apertures, and the number of cycles performed may be varied. If additional micronization is required (or if the preparation will be delivered through a smaller gauge needle [e.g., 27 g]), additional processing can be performed by transferring the 1.2 mm fragmented sample through an even smaller diameter conduit (e.g., 0.8 mm).

In various embodiments, the syringe pump used with processing system 100 is programmable or controlled by a computer, and has sufficient force to achieve a flow rate of 1 mL/min up to 100 mL/min or more when transferring lipoaspirated adipose tissue through the MPD with various diameter conduits. The syringe pump is also capable of providing a short burst of much higher force in order to clear an adipose tissue particle that has become stuck in the flow path. A rapid reversal in the application of force may also be used to remove a blockage. In some aspects, a system controller automatically adjusts between conduits 328(1)-(4).

In an embodiment, the design of the conduits is such that shear force is maximized as the adipose tissue flows through the conduit. For example, the inner surface of the conduit may be textured with slightly raised ridges oriented along the axis of tissue flow to channel the adipose tissue and impart angular momentum to the tissue mass as it moves through the conduit.

In an embodiment, a small mixing chamber is placed between the syringe tip and the inlet of the conduit or pathway such that the tissue flows along a non-restricting channel with ridges oriented perpendicular to the axis of tissue flow to provide additional mixing of the particles in the lipoaspriated adipose tissue sample as it flows through the mixing chamber and into and through the lumen of a conduit, proceeding into the mixing chamber attached to the receiving syringe as the syringe pump delivers physical force to microfragment the lipoaspirated adipose tissue.

In another embodiment, the device can be operated outside of a BSC, with just two sterile breaks, which is far fewer than current microfragmentation kits offer. In this embodiment, a receiving syringe is pre-attached to the inlet port of the MPD, while the other inlet port is sealed by a cap and sterilized as a unit and provided as a sterile kit. The cap is removed from the initial inlet (first sterile break) and the primary syringe containing lipoaspirated adipose tissue is attached. The primary syringe is placed in the syringe holder of the syringe pump, and the receiving syringe is placed in the spring-loaded return device. After processing through the MPD, the syringe with the final, micronized adipose tissue is detached from the MPD and capped (second sterile break).

In another embodiment, the mixing chamber will have one or more large mesh metal frits placed along the flow pathway to aid in providing mixing of the tissue as it flows from the primary syringe through the mixing chamber and into the adjacent conduit. Another mixing chamber with one or more large mesh metal frits will be positioned between the end of the conduit and the inlet of the receiving syringe.

In another preferred embodiment, the connecting port structures have a valve that can divert the flow of micronized adipose tissue to allow for the sterile transfer of a portion of the micronized adipose tissue into a receiving conical tube, which resides in a section of the device that performs cell analysis.

In another preferred embodiment, there is a valve in each connecting port structure that is in communication with a hydrophobic sterile filter that allows for air to be discharged and thereby removed from the adipose tissue present in the syringe.

### WORKING EXAMPLES

Reference will now be made to particular materials and methods utilized by various embodiments of the present disclosure. However, it should be noted that the materials and methods presented below are for illustrative purposes only and are not intended to limit the scope of the claimed subject matter in any way.

### Example 1: Selecting the Spring

The first step in assessing the performance of the spring-loaded return device was to evaluate a series of springs that varied in their degree of compressibility. The experimental design for assessing the suitability of a particular spring is as follows:

Load 20 mL of water into the primary syringe and install it in the syringe pump (NE-1000, SyringePump.com) syringe housing.

Load a spring (or if the spring is shorter, load additional moveable plugs and two springs) in the chamber of the spring-loaded return device and re-install the moveable plug so that it is between the receiving syringe plunger flange and the spring.

Insert the receiving syringe barrel flange into the hold-down end of the return device such that the receiving syringe plunger flange is in contact with the moveable plug. The receiving syringe plunger seal will be positioned at the opposite end of the syringe barrel such that the plunger seal is in contact with the end of the syringe to which is affixed a port of the MPD.

Activate the NE-1000 syringe pump to initiate an infuse stroke of water at a particular flow rate.

Monitor the syringe pump mechanism for any sounds of mechanical stress, such as “clicking”, which might be sounded prior to the syringe pump stalling out and stopping the infusion. The syringe pump is designed to “stall” or stop when the resistance to infuse exceeds a safety margin established for the syringe pump. After completing the infuse stroke, initiate the withdraw stroke.

### Results for Mechanical Processing Experiments

Henke-Sass-Wolf (HSW) syringes with 50/60 mL capacity were used for all spring evaluations, due to the large inner diameter of the syringe, which permitted the use of a 34.15 mL/min flow rate—the highest flow rate available on the syringe pump. Springs were loaded into the spring-loaded return device and the evaluation was initiated as described above.

TABLE 1
Outcomes of evaluating springs with different compressibility

| Spring | | Connector | Flow Rate | |
|---|---|---|---|---|
| Identification | Date | (mm) | (mL/min) | Outcome |
| LC085N08 S316 | Mar. 20, | 2.4 | 34.15 | clicking on infuse |
| | 2022 | | | |
| LC072L 12S | Apr. 3, | 2.4 | 17.07 | clicking on infuse |
| | 2022 | | 34.15 | clicking on infuse |
| LC063L 12S | Apr. 3, | 2.4 | 17.07 | no clicking on |
| | | | | infuse |
| | 2022 | | 34.15 | no clicking on |
| | | | | infuse |
| LC050K 10S | Apr. 20, | 1.2 | 34.15 | no clicking on |
| | | | | infuse |
| | 2022 | 2.4 | 34.15 | no clicking on |
| | | | | infuse |

Becton Dickinson 50/60 mL syringes were used to assess the appropriate spring for the kdScientific Syringe Pump (Model 410), which operates at approximately twice the flow rate of the NE-1000 syringe pump. Spring LC085N08 316 was evaluated and found to perform without stalling when transferring water through the MPD conduits of 2.4 mm, 1.4 mm. and 1.2 mm at 70 mL/min on both infuse and withdraw strokes.

### Example 2: Mechanically Processing Lipoaspirated Adipose Tissue with Spring-loaded Return Device

Standard lipoaspirate (lipoaspirate obtained by the use of a liposuction cannula) was received and stored at 4° C. The lipoaspirate was received in 15 10 mL syringes. The syringes were stored vertically, but very little fluid collected at the tips of the syringes. Each evaluation was performed with HSW 50/60 syringes for both the primary and receiving syringe and used the LC050K 10S springs in the spring-loaded return device. The syringe pump was programed to accommodate the HSW 50/60 mL syringes, which had a maximum flow rate of 34.15 mL/min. The primary syringe was loaded with 20.4 g with an approximate volume of 22 mL (Processing Sequence Number 1) and 21.27 g with an approximate volume of 23 mL (Processing Sequence Number 2) lipoaspirated adipose tissue.

TABLE 2
Mechanical Processing of Lipoaspirated Adipose
Tissue with Spring-loaded Return Device

| Processing | | | Infuse/Withdraw | |
|---|---|---|---|---|
| Sequence | Connector | Cycle | Flow Rate | |
| Number | (mm) | Number | (mL/min) | Outcome |
| 1 | 2.4 | 1 | 10.0 | no clicking |
| | | 2 | 34.15 | no clicking |
| | | 3 | 34.15 | no clicking |
| | 1.4 | 1 | 34.15 | no clicking |
| | | 2 | 34.15 | no clicking |
| | | 3 | 34.15 | no clicking |
| | 1.2 | 1 | 34.15 | no clicking |
| | | 2 | 34.15 | no clicking |
| | | 3 | 34.15 | no clicking |
| 2 | 2.4 | 1 | 10.0 | no clicking |
| | | 2 | 34.15 | no clicking |
| | | 3 | 34.15 | no clicking |
| | 1.4 | 1 | 34.15 | no clicking |
| | | 2 | 34.15 | no clicking |
| | | 3 | 34.15 | no clicking |
| | 1.2 | 1 | 34.15 | no clicking |
| | | 2 | 34.15 | no clicking |
| | | 3 | 34.15 | Immediate stall at |
| | | | | start of infuse |
| | | | | stroke, reversed |
| | | | | flow briefly and |
| | | | | restarted and ran |
| | | | | without incident |
| | | 4 | 34.15 | no clicking and no |
| | | | | stalling |

During each withdraw stroke for each of the conduits, the receiving syringe “hops” on occasion, as if there is some slight impediment in the smooth passage of the receiving syringe seal. Similar resistance to a smooth movement of the syringe seal occurs during the loading of the syringe with lipoaspirate when expelling air from the HSW 50/60 syringe, which suggests that the thin-walled HSW syringe might not be structurally rigid enough for a smooth glide of the syringe seal. The “hops” of the receiving syringe more often occur on the withdraw stroke. The stall that occurred just at the initiation of the third cycle through the 1.2 mm connector with the Processing Sequence Number 2 processing experiment might be explained by the sticking of the receiving syringe seal momentarily rather than a clogged connector. After completing the third cycle, a fourth cycle was immediately initiated and proceeded without clicking or stalling on both the infuse and withdraw strokes.

### Characterization of the Micronized Lipoaspirated Adipose Tissue

### Tissue Digestion and Particle/Cell Analysis

The cells in micronized adipose tissue preparations are embedded in an adipose tissue matrix that includes connective tissue and adipocytes. The widely published approach to analyzing cells in adipose tissue is to digest the tissue with an enzyme(s), typically for 0.5-1.5 hours, and frequently at 37° C. The digest is composed of one or more enzymes diluted in a diluent (typically Phosphate Buffered Saline, or a version of tissue culture medium, e.g., Dulbecco's Modified Eagle Medium with or without fetal bovine serum [FBS]). After the digestion, the released nucleated cells can be assessed for cell count and viability. One approach to assess the viability of cells present in micronized adipose tissue is by incubating the micronized tissue with fluorescent vitality dyes, with visualization performed on a confocal immunofluorescence microscope. Another approach is to count the “particles” released following the enzymatic digestion. Impedance-based particle counting instruments will count the various types of released particles and can assign a particle diameter to the various particles detected.

The inventive enzymatic-based digestion method was adapted to be completed within 15 minutes. Initiation of the inventive enzymatic-based digestion method begins with transferring an amount of the micronized adipose tissue preparation to a pre-weighed conical tube. A volume of tissue, ranging from approximately 1 mL up to 25 mL, is placed in the pre-weighed conical tube. The tube with micronized tissue is weighed, the tared tube weight is subtracted and the resulting mass of micronized adipose tissue in the conical tube is noted, along with the approximate volume of the micronized tissue. The tube with the tissue is placed in the water bath to equilibrate to the digestion temperature. The volume of Enzyme Digestion Solution (EDS) prepared will be equal to the volume of the micronized adipose tissue to be digested, and should be prepared in a sterile, 50 mL conical tube. The amount of collagenase to be added will range from 1 mg/mL, 2 mg/mL, 5 mg/mL, 10 mg/mL, 15 mg/mL, 20 mg/mL, 25 mg/mL, 30 mg/mL, 40 mg/mL or more in the EDS. A sufficient volume of rehydrated Collagenase stock solution that has been stored frozen will be thawed and added to the EDS conical tube. A volume of rehydrated Neutral Protease stock solution that has been stored frozen will be thawed, and if used, added to the EDS conical tube. A sufficient amount of a stock solution of CaCl₂ in Dulbecco's Phosphate Buffered Saline (DPBS) is added to bring the final concentration of CaCl₂) in the EDS to 0.1 mM. A sufficient volume of DPBS is added to the EDS conical tube to bring the total volume in the EDS conical tube to the target volume of the EDS. Once the EDS has been prepared, the EDS conical tube is capped and placed in the water bath to equilibrate to the digestion temperature. After the EDS and micronized adipose tissue preparation have equilibrated to the digestion temperature, the contents of the EDS conical tube is transferred to the micronized adipose tissue conical tube, re-capped and shaken by hand vigorously for 10 shakes (back and forth defines a “shake”), 15 shakes, 20 shakes, 30 shakes, 40 shakes or more. Alternative methods of shaking in a water bath include the use of a shaking water bath, or the suspension of the tissue digestion tube in a holder beneath the water level of the water bath, with the other end of the holder attached to a device that provides a controlled side-to-side or orbital motion shaking (e.g., a vortexer that has been adapted with a fitting that can securely hold the digestion tube holder assembly). The digestion tube is removed from the water bath at periodic intervals and shaken vigorously by hand as indicated previously. The number of shaking events ranges from 3 (every five minutes) to 5 (every 2.5 minutes) or more, until a total of 15 minutes has elapsed, after which, the digestion tube is removed, and shaken vigorously by hand as indicated previously. DPBS is added to the digestion tube to fill the fluid volume to approximately 50-mL mark on the conical tube. The contents are mixed and the tube is centrifuged at 1900 rpm (740×g) for five minutes. After the centrifugation step, there will be three zones of interest. At the bottom of the conical tube are released particles/cells in a “pellet”, including red blood cells (RBCs), and depending on the number of RBCs present, a red ring might be visible. Immediately above the pellet is the infranatant and above the infranatant is a floating tissue/lipid layer. Several means are used to remove the upper two zones of material, while leaving the pellet intact. It is possible to pour off the two upper zones, but sometimes the pellet is loose and could be displaced while pouring off the upper two zones. A second approach is to aspirate the upper two zones with a pipette and a Pipet Aid (or equivalent pipette controller). A variation on the aspiration approach involves the use of a vacuum, a collection container and tubing attached to a pipette. Aspiration of the two zones above the pellet is performed to leave approximately five mL of infranatant above the pellet. Once the two zones have been removed, the pellet can be resuspended in a small volume of DPBS (if the pour-off method is used) or in the remaining fluid with the use of a transfer pipet. Using the same transfer pipet, the resuspended cell volume is transferred to a conical tube fitted with a cell strainer. After removing the cell strainer, the tube is filled with DPBS, capped and mixed. The cell suspension is centrifuged at 1900 rpm for five minutes. After the centrifugation, the supernatant is removed by one of the techniques described above. The pelleted cells are resuspended in a small volume of DPBS (ranging from 0.3 mL, 0.5 mL, 1.0 mL, 2.0 mL, 5 mL, 10 mL or more), which is denoted as the released particle/cell preparation.

The particles in the released particle/cell preparation are assessed for viability and particle concentration by the use of a semi-automated cell counter. MOXI GO II has been used for this purpose, but other semi-automated cell counters (e.g., NucleoCounter NC-200) also could be used.

In an embodiment, after obtaining the particle count and nucleated cell viability, the released particle preparation is further concentrated by centrifugation, such that the resulting volume is approximately 0.1 mL, 0.2 mL, 0.3 mL up to 0.5 mL. This highly concentrated released particle preparation is assessed for cell type and number on a hemoanalyzer instrument like the Sysmex XN-350.

In an embodiment, the PMT filter of the MOXI GO II is adjusted to a 561 nm/LP filter, which can detect a variety of fluorescent molecules, including Phycoerythrin (PE). The PE-tag is used with CD marker reagents like CD44, which detects stromal progenitor cells. Other CD markers of interest include CD31 (endothelial progenitor cells), CD45 (adult hematopoietic cells, except RBCs and platelets), CD49f (stromal progenitor cells), and other CD markers known in the art. For CD marker analysis, the concentrated released particle/cell preparation is incubated with the CD marker reagent on ice and in the dark for 30 minutes. While an aliquot of each of the CD marker reagents is being incubated with an aliquot of the concentrated released particle/cell preparation, the PE-compatible (561 nm/LP) PMT filter should be inserted and the instrument calibrated. The samples incubated with the PE-CD markers are assessed with the Open Flow module to determine the expression frequency of each CD-marker incubated.

In an embodiment, fluorogenic substrates for various enzymatic activities that can be detected by the 525/45 nm PMT filter, the 561 nm/LP or the 646 nm/LP PMT filter on the MOXI GO II instrument are incubated with aliquots of the concentrated released particle/cell preparation and/or the post-digestion floating adipose tissue layer, which contains adipocytes, as well as other cell types. An aliquot of the concentrated released particle/cell preparation is diluted into a culture medium, like DMEM low glucose with 10% FBS that contains the fluorogenic substrate(s), and placed into a humidified, 5% CO2 incubator. The cells are allowed to metabolize the fluorogenic substrate for a sufficient time and then are assessed for the level of the fluorescence in the test aliquot. The adipocyte-rich floating adipose tissue is collected after completing the enzymatic digestion step, and processed to obtain an isolated adipocyte fraction by methods known in the art. The adipocyte fraction is mixed with fluorogenic substrates for enzymatic activities highly associated with adipocytes (e.g., adipocytic-associated aminopeptidases, adipocytic-associated triacylglyceride lipases). The adipocyte suspension is incubated with the fluorogenic substrate at elevated temperature and the resulting fluorogenic signal is determined after a washing step on the MOXI GO II fitted with the appropriate PMT filter.

In an embodiment, the micronized adipose tissue preparation prepared in the micronization section of the inventive device is transferred to the enzymatic digestion section of the inventive device, wherein the steps established for the rapid digestion conditions are performed through the use of a computer-assisted controller that is able to transfer pre-loaded enzymatic digestion solutions, wash solutions, control agitation in a heated incubator, transfer the post-digestion suspension through a means to separate the released cells (e.g., by centrifugation or microfluidic device separation), resulting in a semi-automated production of a released particle/cell preparation, which can be characterized either with an on-board impedance based microfluidic device or processed externally as described herein by the use of the MOXI GO II or comparable cell analyzer.

In an embodiment, a stand-alone apparatus is used to process the digested adipose tissue in which a vacuum assembly (tubing, vacuum, collection container) is used with a moveable aspiration piece (aspiration head) that holds a pipette or other aspiration means in a fixed position relative to the 50 mL conical tube from which the fluid needs to be removed without disturbing the pelleted cells at the bottom of the conical tube. In operation, after centrifugation, the conical tube is placed in a holder and the aspiration head with the suctioning tube is brought down to remove the fluid. There is a physical limit for the depth of penetration of the suctioning tube to leave a sufficient volume of the fluid so as not to disturb the pelleted particles/cells.

### Physical Characterization of the Micronized Adipose Tissue Preparation

There is no systematic publication of size distributions of the particles in a micronized adipose tissue preparation. As a part of the characterization of the micronized adipose tissue preparations produced with the inventive device, an assessment of the size distribution of the micronized adipose tissue preparations is established with, for example, an instrument that performs a laser light scattering of the micronized adipose tissue preparation. Horiba Corporation manufactures an instrument that incorporates laser light particle scattering technology that has a particle range of 10 nm through 5 mm. Lensless holographic imaging also offers size distribution assessments of particles in suspension.

### Example 3: Assessing the Number of Manual Shakes During Digestion

Two sets of BodyJet lipoaspirate samples were digested with 20 mg/mL DE800 Collagenase in the EDS, with no additional Neutral Protease added. The digestions were performed at 37° C. for 15 minutes with 7 manual shaking steps at 0, 2.5 min, 5 min, 7.5 min, min, 12.5 min, and 15 min. The released particles/cells were processed as described above, with the final total particle number divided by the mass of the BodyJet lipoaspirate, micronized adipose tissue preparations.

TABLE 3
Comparison of Viability % and Particles/g
Yield for 20 shakes or 40 shakes

| Sample | Number of | Viability | Particles/g of | Average |
|---|---|---|---|---|
| ID | Shakes | (%)* | adipose tissue | Particles/g |
| GL-001 | 20 | 100 | 480,000 | 680,637 |
| GL-002 | | 100 | 1,145,161 | |
| GL-003 | | 100 | 23,626 | |
| GL-004 | | 100 | 1,148,014 | |
| GL-005 | | 100 | 607,386 | |
| GL-006 | 40 | 99.6 | 396,226 | 550,014 |
| GL-007 | | 100 | 1,304,878 | |
| GL-008 | | 100 | 220,126 | |
| GL-009 | | 100 | 363,402 | |
| GL-010 | | 100 | 465,438 | |

*Viability % refers to the level of autofluorescence of the sample, which is measured in the absence of a vital stain like Propidium Iodide.

The assessment of the number of shakes showed that increasing the number of shakes from 20 to 40 didn't adversely alter the autofluorescence (listed as “viability %”) of the released particles/cells. The average particles/g decreased with the 40-stroke set of samples, but this might reflect the fact that the 20-stroke set had two high-yield samples, which clearly biased the average particles/g. The number of shakes was increased to 40 for all subsequent processing, since there was no adverse impact on the autofluorescence of the released cells.

### Example 4: Assessing the Use of Collagenase

The collagenase used for all digestions is DE800 from VitaCyte (Indianapolis, IN). This is a premixed combination of highly purified Collagenase isolated from Clostridium histolyticum and highly purified Neutral Protease isolated from Paenibacillus polymyxa. The assessment of DE800 concentration was performed at 37° C. with 40 shakes during 7 manual shaking steps at 0, 2.5 min, 5 min, 7.5 min, 10 min, 12.5 min, and 15 min.

TABLE 4
Comparison of 20 mg/mL and 40 mg/mL
Collagenase concentrations in the EDS

| | DE800 | | | |
|---|---|---|---|---|
| Sample | Concentration | Viability | Particles/g of | Average |
| ID | (mg/mL) | (%)* | adipose tissue | Particles/g |
| GL-011 | 20 | 99.6 | 396,226 | 550,014 |
| GL-012 | | 100 | 1,304,878 | |
| GL-013 | | 100 | 220,126 | |
| GL-014 | | 100 | 363,402 | |
| GL-015 | | 100 | 465,438 | |
| GL-016 | 40 | 100 | 893,617 | 596754 |
| GL-017 | | 100 | 359,684 | |
| GL-018 | | 100 | 630,675 | |
| GL-019 | | 100 | 395,613 | |
| GL-020 | | 100 | 688,685 | |
| GL-021 | | 100 | 612,252 | |

*Viability % refers to the level of autofluorescence of the sample, which is measured in the absence of a vital stain like Propidium Iodide.

There was no adverse impact on autofluoresence (indicated as “Viability %”) with the use of 40 mg/mL DE800 in the EDS during the 15-minute incubation at 37° C. with 40 shakes. There also was an increase in the average Particles/g yield of approximately 46,000 particles/g, which indicates that the digestion process can be influenced by the concentration of the DE800 as one means for increasing the yield of particles per gram of tissue for a fixed incubation interval of 15 minutes.

### Example 5: Assessing the Use of Different Temperatures for Digestion

A set of BodyJet lipoaspirated adipose tissue samples was digested at 37° C. and another set of BodyJet lipoaspirated adipose tissue samples was digested at 42° C. with 20 mg/ml DE800 in the EDS, with no additional Neutral Protease, and with 40 shakes during 7 manual shaking steps at 0, 2.5 min, 5 min, 7.5 min, 10 min, 12.5 min, and 15 min.

TABLE 5
Digestion of Lipoaspirated Adipose Tissue at 37° C. and
42° C. with 20 mg/mL DE800 Collagenase in the EDS

| Sample | Digestion | Viability | Particles/g of | Average |
|---|---|---|---|---|
| ID | Temperature | (%)* | adipose tissue | Particles/g |
| GL-011 | 37° C. | 99.6 | 396,226 | 550,014 |
| GL-012 | | 100 | 1,304,878 | |
| GL-013 | | 100 | 220,126 | |
| GL-014 | | 100 | 363,402 | |
| GL-015 | | 100 | 465,438 | |
| GL-022 | 42° C. | 100 | 1,218,286 | 755,827 |
| GL-023 | | 100 | 463,918 | |
| GL-024 | | 99.0 | 535,738 | |
| GL-025 | | 100 | 277,092 | |
| GL-026 | | 100 | 650,000 | |
| GL-027 | | 100 | 1,389,930 | |

*Viability % refers to the level of autofluorescence of the sample, which is measured in the absence of a vital stain like Propidium Iodide.

Conventionally, tissue digestion temperatures have been in the range of 35-37° C. Contrary to the conventional temperature range, the use of 42° C. during the digestion incubation unexpectedly didn't have an adverse impact on the autofluoresence (indicated as “Viability %”) of the released particles, while also producing an average of approximately 200,000 particles/g higher yield compared to the yield with the digestion temperature of 37° C. The optimal temperature range for Collagenase activity is reported to be 35-37° C. (VitaCyte website), which makes the higher particles/g yield obtained at 42° C. compared to 37° C. an unexpected outcome.

### Example 6: Use of Various Enzymatic Compositions During Digestion of MEAT

Several compositions of enzyme digestion solution (EDS) have been assessed for the efficiency of releasing particles (e.g., nucleated cells and RBCs) from microfragmented fat tissue (MFAT). Enzymatic digestion of MFAT results in a single particle suspension identified as the stromal vascular fraction (SVF). Three enzymes have been evaluated: Collagenase, Hyaluronidase and Neutral Protease. The collagenase-containing preparation (DE800) is a custom combination of collagenase activity and neutral protease activity produced by VitaCyte, Inc. (Indianapolis, IN). Thus, one digestion condition consists of DE800 without any additional enzymes. DE800 with either 4,400 or 16,700 NPU (neutral protease units)/mL additional neutral protease activity (BP Neutral Protease, VitaCyte) was assessed. DE800 with 390 IU of Hyaluronidase (Sigma Aldrich, St. Louis, MO) was assessed. Finally, a combination of DE800 at 20 mg/mL, 16,700 NPU/mL Neutral Protease activity and 390 IU/mL of Hyaluronidase activity was assessed. The parameter of Total Particles/g of adipose tissue digested was determined for BodyJet samples when incubated at 42° C., with 40 shakes at 0, 2.5, 5, 7.5, 10 12.5 and 15 minutes. Each EDS contained 0.1 mM CaCl₂). A set of BodyJet samples was digested with each type of EDS and the resulting SVF samples were analyzed on the Moxi GO for total particles, which was divided by the mass of the digested adipose tissue to obtain an estimate of Total Particles/g adipose tissue. The average Total Particles/g adipose tissue is shown for the EDS compositions tested in Table 6.

TABLE 6
Particles/g of BodyJet Samples Released
with Different Combinations of Enzymes

| | | | Total | |
|---|---|---|---|---|
| | Average | | Particles/g | |
| | Donor | | Average | Range |
| Enzyme Digestion Solution | Age | Sample | (SD) | (min/max) |
| Composition | (yr) | Size | (×10⁶) | Particles/g |
| DE800 Collagenase-only | 74 | 11 | 3.18 (2.13) | 1.11/6.77 |
| DE800/Hyaluronidase/BP | 76 | 12 | 3.18 (1.91) | 1.00/7.67 |
| Neutral Protease (16,700 | | | | |
| NPU/mL) | | | | |
| DE800/Hyaluronidase | 59.3 | 6 | 3.72 (3.46) | 1.9/11.4 |
| DE800/BP Neutral Protease | 72 | 7 | 3.04 (1.01) | 1.32/4.0 |
| (4,400 NPU/mL) | | | | |

Use of the DE800 Collagenase/Neutral Protease product gives comparable averaged Total Particles/g of adipose tissue compared to the other EDS compositions. While the DE800/Hyaluroidase set of samples had a higher average Total Particles/g value, it also had the highest standard deviation and the lowest average age of the donor BodyJet samples, which might have played a role in the apparent increased particle yield per gram of adipose tissue observed with the addition of Hyaluronidase to the EDS. All combinations of enzymes used for the EDS have provided substantial particle release for a 15-minute incubation period.

### Example 7: Use of Impedance Measurements for MFAT Characterization

Particles, like RBCs or white blood cells (WBCs), can be counted when suspended in an ionic fluid, like Phosphate Buffered Saline (PBS), based on a change in conductivity as the particles move through an aperture, resulting in a momentary change in impedance. The Moxi GO instrument (OrFlo Technologies, Ketchum, ID) has been used to perform an impedance measurement of the particles present in the SVF samples obtained from the MFAT preparations. In addition, the Moxi GO has a capability of detecting fluorescent reagents, including vitality stains like Propidium Iodide (PI), Acridine Orange (AO), Live/Dead Fixable Stain (L/D), as well as CD markers, which typically are antibodies directed against a specific antigen on or in a cell that is uniquely associated with a type of cell. For example, CD235a is used to identify RBCs.

Once an MFAT preparation is obtained, it is treated with DE800 Collagenase, which results in a single particle suspension (SVF) that can be characterized. An aliquot of the SVF is loaded onto a Moxi GO cassette and the cassette is placed in the Moxi GO instrument. An analysis is performed based on impedance that includes a determination of all particles present in the SVF, which includes RBCs, WBCs, and other nucleated cells like adipose-derived stromal cells (ASC). The analysis of an SVF sample obtained from a MFAT preparation was performed and graphed as the size distribution (X-axis is in microns) of the number of particles present in the SVF sample (Y-axis is particle number). The area under each peak represents the nominal particle count for that peak. A two-peak distribution is a frequently observed feature of the SVF produced by the digestion of an MFAT sample, which in this case was obtained with the following enzymatic digestion protocol: 20 mg/mL DE800 Collagenase (VitaCyte, Indianapolis, IN), with 0.1 mM CaCl₂) at 42° C., for 15 minutes with 40 shakes at 0, 2.5, 5, 7.5, 10, 12.5 and 15 minutes. It is known that human RBCs are one type of particle with a nominal diameter of approximately 6 microns (one of the observed peaks). However, the use of the Moxi GO's impedance analysis capability alone doesn't provide an identity of the particles distributed along the X-axis, and without addition of a viability stain, the viability of the particles can't be assessed.

In a clinical setting, there is a need to obtain and characterize the MFAT preparation as quickly as possible, and the time to obtain an impedance-based image once the SVF is available is typically less than one minute.

In an analysis of SVF samples obtained by digesting 23 MFAT preparations (BodyJet MFAT), a 2-peak pattern was observed in all cases, but with some variations. If the left-most peak is identified as “Peak 1” and the right-most peak is “Peak 2”, then it is possible to assess the nominal number of particles under each peak in order to characterize the relationship of the peaks in terms of particle counts, such as “Peak 1 count>Peak 2 count”, or “Peak 2 count>Peak 1 count”. Further characterization is possible by assessing the degree of difference in particle counts between the two peaks. Four histogram profiles of the particles present in SVF samples routinely have been observed when the particle count doesn't exceed the Moxi GO upper limit of 1.75 million particles/mL: Type 1a: Peak 1 particle count is greater than Peak 2 particle count; Type 1b: Peak 1 particle count is much greater (e.g., 2-fold) than Peak 2 particle count; Type 2a: Peak 2 particle count is greater than Peak 1 particle count; and Type 2b: Peak 2 particle count is much greater (e.g., 2-fold) than Peak 1 particle count.

While it is obvious that the peak counts of an SVF sample might be greater than 2-fold, e.g., Type 2b, it isn't always possible to assign the appropriate Peak Profile type for the SVF preparation based on a visual inspection alone. In order to quickly determine the peak profile type for a SVF analysis in impedance mode on the Moxi GO, the following method can be used to obtain an estimate of the total particle count for the sample, and the particle count associated with Peak 2.

Turn on the Moxi GO and select the Cell QC module for impedance analysis.

After digestion and resuspension of the pellet to generate the SVF sample, insert the S-type cassette into the reading chamber of the Moxi GO and initiate alignment of the instrument.

Load the test aliquot (typically 62 μL) from the SVF sample into the test well of the cassette and initiate the analysis.

When the analysis is complete, a scattergram will appear on the screen with two blue dots on the top edge and the histogram of the peaks will be outlined in red. A box will appear that gives the operator the option to set the position of the gates manually.

The manual gate setting option is selected.

On the screen, touch and hold the blue dot at the upper left edge of the scattergram display and move the dot all the way to the left. This will set the left gate at the lowest detectable particle size allowed by the Moxi GO, as indicated by “0% debris” shown in the “Live Cells” display box.

Record the total concentration of “cells” (“cells/mL”) shown in the “Live Cells” display box.

Adjust the position of the left gate by touching the blue dot at the upper left edge of the scattergram display and move it toward the right until it is approximately at the minimum of the red line between the two peaks.

Record the total concentration of “cells” (“cells/mL”) shown in the “Live Cells” display box, which corresponds to the concentration of the particles present in Peak 2.

Divide the particle concentration for Peak 2 by the total particle concentration, which will give a fractional number for P2 (P2f). P1f is obtained from (1-P2f).

The following rules apply for determining the profile type of the SVF:

$$If P 2 f > P 1 f then the peak type is 2. \\ If P 1 f > P 2 f then the peak type is 1. \\ If P 2 f / 2 > P 1 f then the peak type is 2 b . \\ If P 2 f / 2 < P 1 f then the peak type is 2 a . \\ If P 1 f / 2 > P 2 f then the peak type is 1 b . \\ If P F 1 / 2 < P 2 f then the peak type is 1 a .$$

Note: The rules for assigning the subtype of the peak profile as “a” or “b” are based on a 2-fold difference in particle count between the two peaks. Other fold differences could be adopted (e.g., 1.5-fold, 3-fold).

In a survey of 23 SVF samples of MFAT preparations digested with 20 mg/mL DE800 with 0.1 mM CaCl₂) at 42° C. with 40 shakes at 0, 2.5, 5, 7.5, 10, 12.5 and 15 minutes the distribution of Profile Types is shown in Table 7 with a criterion of a 2-fold difference.

TABLE 7
Summary of SVF Profile Types for 23 SVF Samples
Obtained by Digestion of MFAT Preparations

| Profile Type | Number | Percentage (%) |
|---|---|---|
| 1a | 4 | 17.4 |
| 1b | 10 | 43.5 |
| 2a | 7 | 30.4 |
| 2b | 2 | 8.7 |

Approximately 44% of the profiles are Type 1b, 30% are Type 2a, 17% Type 1a, while Type 2b is the least frequently observed at 9%. In addition to the Peak Profile parameter, the impedance mode of the Moxi GO also provides an estimate of the total number of particles for the SVF sample by recording the total “cells/mL” value displayed on the Moxi GO and subsequently multiplying that value times the volume of the SVF. The mass of MEAT tissue that was digested to yield an SVF sample is recorded, which means that an additional parameter for “total particles/g MFAT digested” could be reported. Furthermore, the particle counts associated with each peak in the histogram also could be divided by the mass of the MFAT tissue digested resulting in two additional parameters that characterize the SVF for tracking purposes: “Peak 1 particles/g MFAT digested” and “Peak 2 particles/g MFAT digested”.

Thus, it is possible without any additional processing of the SVF sample to perform an impedance-based analysis of the particles present in an SVF sample prior to return of the patient's MFAT for treatment, which results in multiple parameters that are unique to the patient's MFAT preparation: Peak Profile Type (e.g., 1a, 1b, 2a, 2b) (impedance mode); Total Particles Released During Digestion (impedance mode); Total Particles Released/gram of MFAT digested (“Total Particles/g”); Peak 1 Particles Released/gram of MFAT digested (“P1 Particles/g”); Peak 2 Particles Released/gram of MFAT digested (“P2 Particles/g”).

One or more of these parameters can be entered into the patient's medical record and/or added to the patient's data maintained on a registry, which reflects a property of the MFAT preparation that the patient received that might over time show a correlation to therapeutic benefit for all patients in the registry.

### Example 8: Use of Fluorescent Reagents to Assess Viability of Nucleated Cells

Use of the Moxi GO without the presence of a fluorescent agent like PI will provide a particle count as described in Example 7, but will not assess the viability of nucleated cells present in the SVF. Thus, when no fluorescent viability stain is present, the result determined by the Moxi GO for “viability” is a reflection of the autofluorescence of the particles present in the SVF sample. Freshly isolated cells like granulocytes are known to have a higher autofluorescence, while the autofluorescence of stromal/progenitor cells found in SVF preparations is poorly characterized. Autofluorescence of cultured mesenchymal stromal cells increases as the cells age in culture due to senescence. Thus, the level of autofluorescence detected is determined by the cells present in the SVF. Throughout the assessment of various conditions (e.g., enzyme concentrations, enzyme combinations, temperature of digestion) on the digestion of MFAT preparations to yield SVF samples, the autofluorescence has been low, as indicated by a reported “viability” usually of 99% to 100%. This suggests that the various conditions used during the digestion of MFAT preparations haven't resulted in changes to the autofluorescence of the particles released with the various digestion protocols.

One of the complications in obtaining an accurate viability assessment (viable nucleated cells/total nucleated cells) is that the Moxi GO will use all particles detected as the value in the denominator, which will include RBCs. Thus, it is necessary to remove the RBCs from the viability analysis. There are two ways to address the RBC artefact: 1) Use an RBC Lysis Buffer with an aliquot of the SVF, which will lyse substantially all of the RBCs, and then measure the viability; or 2) Use the RBC marker CD235a to obtain a count of the CD235a+ particles with the fluorescence mode of the Moxi GO and subtract this result from the total particles measured in impedance mode.

### Method 1: RBC Lysis Buffer

The protocol for using the RBC Lysis Buffer to determine viability of nucleated cells in SVF samples on the Moxi GO is as follows:
Obtain a freshly made RBC Lysis Buffer at 1X strength by diluting the 10X stock Lysis Buffer with Deionized Water.
Centrifuge an aliquot of SVF (e.g., 1 mL) in a microcentrifuge tube at 1900 rpm for 5 min.
Decant the fluid and blot the microcentrifuge tube on a kimwipe to remove excess fluid.
Resuspend cells in 1 ml (1×10⁶ cells) of 1X RBC lysis buffer.
Incubate for 10 min at room temperature.
Centrifuge the treated sample and resuspend in FACS buffer (DPBS, 3% FBS, 5 mM EDTA).
Centrifuge the treated sample and resuspend in either 1 ml DPBS if staining with the Live/Dead fixable dye or in 1 ml FACS buffer if staining with Sytox Green, PI or AO/PI.
Prior to staining the resuspended preparation, transfer an aliquot to an S+ cassette and determine the total particle count after RBC lysis buffer treatment with the impedance mode of the Moxi GO.
Proceed with the staining protocol to assess the percentage of viable cells with the fluorescence mode of the Moxi GO.

### Method 2: Counting the RBCs Present in the SVF

The protocol for using CD235a fluorescent reagent to determine the number of RBCs present in an SVF sample to estimate the viability percentage of nucleated cells is as follows:
Obtain two aliquots of cells (e.g., 1 mL each) from the SVF sample, place in two microcentrifuge tubes and centrifuge at 1900 rpm for 5 min.
Decant the fluid and blot each microcentrifuge tube on a kimwipe to remove excess fluid.
Resuspend the cell pellet in 1 mL of FACS buffer.
Add 200 μL of rat serum and mouse serum (1:1 v: v) blocking solution to both microcentrifuge tubes and incubate at room temperature for 20 min.
Add 5 μL of CD235a reagent to one of the two microcentrifuge tubes with cells and incubate in the dark for 30 min.
Centrifuge the microcentrifuge tubes at 1900 rpm for 5 min. Decant the fluid and blot each microcentrifuge tube on a kimwipe to remove excess fluid.
Resuspend pellets in 1 mL FACS buffer.
Assess the number of CD235a+ particles in the preparation with the CD235a stained cells, and also the total number of particles in the other microcentrifuge tube.
Add 2-drops of the PI vital stain to the microcentrifuge tube without CD235a stained cells and incubate in the dark for 15 minutes at room temperature.
Select the Cell QC module on the Moxi GO and confirm that the 561 nm/LP PMT filter is available.
Transfer an aliquot of the PI-stained cell sample to an S+ cassette and analyze for “live cell” and “dead cell” numbers, and estimate the total number of particles.
Subtract the number of CD235a+ particles (assume 1 mL volumes to get number of particles) obtained in the PI-stained sample from the total number of particles obtained for this sample prior to adding the PI stain to obtain an estimate of the total number of nucleated cells.
Divide the “live cell” count obtained in the PI-stained sample by the total particle number after subtracting the RBC count.
Multiply the fraction obtained in the previous step by 100 to obtain the viability % of nucleated cells in the SVF sample.
Flow Cytometric Analysis of SVF Obtained from MFAT Produced with the MPD

The MPD was used to generate an MFAT preparation from an abdominal lipoaspirate sample, which was digested with a collagenase-containing EDS to yield an SVF that was analyzed on a flow cytometer. Beads with the approximate diameters of 3, 6 and 10 μm were included to provide a calibration of the forward scatter positions of the Endothelial progenitor cells, Pericytes and Adipose Stromal Cells.

Based on the forward scatter positions of the 3 and 6 μm bead preparations, the three types of progenitor cells are located between the 3 and 6 μm bead positions, with the Adipose Stromal Cells also appearing in a cluster above the 6 μm bead position. The presence of progenitor cells near the 3 μm bead position suggests that the Peak 1 particles, which also are near the 3 μm position as determined by impedance in the Moxi GO, could be associated with progenitor cells identified by flow cytometry, although the positions of the cell types with fly cytometry are based on the forward scatter of 3, 6, and 10 μm beads. The presence of Adipose Stromal Cells in a position on either side of the 6 μm marker might be represented by particles found in Peak 2 area (i.e., straddling the 6 μm zone) after RBC lysis, which eliminates RBCs, but not nucleated cells like Adipose Stromal Cells.

### Example 9: MFAT Obtained by Manual Processing of Lipoaspirate Through the MPD

A lipoaspirate sample obtained from the abdominal region was processed with the MPD device by manual means. The primary syringe was attached to one of the ports of the MPD device and the receiving syringe was attached to the other port. The 2.4 mm conduit was selected and 10 cycles of transfer between the two syringes were completed without clogging. The 1.4 mm conduit was selected and 10 cycles of transfer between the two syringes were completed without clogging. The 1.2 mm conduit was selected and 10 cycles of transfer between the two syringes were completed with three instances of clogging occurring during the initial cycles, which were cleared, allowing the completion of the remaining cycles.

The MFAT obtained with the MPD device was digested with collagenase (1%, Sigma Aldrich, St. Louis, MO), at 37° C. with agitation. The resulting SVF was characterized by flow cytometry (Beckman Coulter DxFlex 3-laser flow cytometer) with the DuraClone (Beckman Coulter, Miami, FL) set of CD marker fluorescent reagents. The cell types and their frequencies observed in the SVF are shown in Table 8.

TABLE 8
Cell types and their frequencies observed in
the SVF obtained after digesting the MFAT obtained
by processing lipoaspirate with the MPD

| | Frequency |
|---|---|
| Cell Type | (% of viable cells) |
| Leukocytes | 9.5 |
| Pericytes | 4.6 |
| Endothelial Progenitor Cells | 82.1 |
| Adipose Stromal Cell (ASC) | 2.8 |

The viability of the SVF nucleated cells was determined to be 85.5% viable nucleated cells based on the flow cytometric analysis.

### Example 10: MFAT Obtained by Syringe Pump-based Processing of Lipoaspirate Through the MPD

A lipoaspirate sample was processed through the MPD. The lipoaspirate was collected from the abdomen with the use of a Mercedes-type cannula. A total of approximately 9 mL was provided in two 10 mL syringes. The lipoaspirate was washed once at the clinic. The lipoaspirate sample was loaded into a 50 mL primary syringe, which was placed in the syringe holding blocks of the kdScientific Syringe Pump Model 410. The receiving syringe was placed in the spring-loaded return device and attached to one of the MPD Luer-lok ports. The Primary syringe was attached to the other Luer-lok port of the MPD. The top knob of the MPD was rotated to bring the 2.4 mm conduit into position. The syringe pump was programmed to perform a single cycle of Infuse/Withdraw (I/W), and the flow rate was set at 35 mL/min for both the Infuse and Withdraw strokes. The cycle was initiated. No stalling occurred during the first cycle at 35 mL/min, so the remaining cycles were performed at 70 mL/min, without stalling. The 1.4 mm conduit was placed into position, and the sample was processed on “Continuous” mode at 70 mL/min for 10 cycles, without stalling. The 1.2 mm conduit was placed into position and the first I/W cycle was run at 70 mL/min. During the 10 I/W cycles through the 1.2 mm conduit, it seemed as if there was “back pressure” and occasionally there was evidence that cavitation (introduction of air into a syringe) was occurring. However, the 10 cycles were completed without stalling.

The micronized fat tissue (MFAT) was digested with an Enzyme Digestion Solution composed of 20 mg/mL DE800/390 IU Hyaluronidase/16,700 NPU Neutral Protease, with 0.1 mM CaCl₂), at 42° C. with 40 shakes at 0, 2.5, 5, 7.5, 10, 12.5 and 15 minutes. The resulting SVF was analyzed on the Moxi GO in impedance mode, which showed a Peak Profile of Type 2a and 2 million particles/g of tissue digested.

Although various embodiments of the present disclosure have been illustrated in the accompanying Drawings and described in the foregoing Detailed Description, it will be understood that the present disclosure is not limited to the embodiments disclosed herein, but is capable of numerous rearrangements, modifications, and substitutions without departing from the spirit of the disclosure as set forth herein.

The term “substantially” is defined as largely but not necessarily wholly what is specified, as understood by a person of ordinary skill in the art. In any disclosed embodiment, the terms “substantially”, “approximately”, “generally”, and “about” may be substituted with “within [a percentage] of” what is specified, where the percentage includes 0.1, 1, 5, and 10 percent.

The foregoing outlines features of several embodiments so that those skilled in the art may better understand the aspects of the disclosure. Those skilled in the art should appreciate that they may readily use the disclosure as a basis for designing or modifying other processes and structures for carrying out the same purposes and/or achieving the same advantages of the embodiments introduced herein. Those skilled in the art should also realize that such equivalent constructions do not depart from the spirit and scope of the disclosure, and that they may make various changes, substitutions, and alterations herein without departing from the spirit and scope of the disclosure. The scope of the invention should be determined only by the language of the claims that follow. The term “comprising” within the claims is intended to mean “including at least” such that the recited listing of elements in a claim are an open group. The terms “a”, “an”, and other singular terms are intended to include the plural forms thereof unless specifically excluded.

Depending on the embodiment, certain acts, events, or functions of any of the algorithms described herein can be performed in a different sequence, can be added, merged, or left out altogether (e.g., not all described acts or events are necessary for the practice of the algorithms). Moreover, in certain embodiments, acts or events can be performed concurrently, e.g., through multi-threaded processing, interrupt processing, or multiple processors or processor cores or on other parallel architectures, rather than sequentially. Although certain computer-implemented tasks are described as being performed by a particular entity, other embodiments are possible in which these tasks are performed by a different entity.

Conditional language used herein, such as, among others, “can”, “might”, “may”, “e.g.”, and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

While the above detailed description has shown, described, and pointed out novel features as applied to various embodiments, it will be understood that various omissions, substitutions, and changes in the form and details of the devices or algorithms illustrated can be made without departing from the spirit of the disclosure. As will be recognized, the processes described herein can be embodied within a form that does not provide all of the features and benefits set forth herein, as some features can be used or practiced separately from others. The scope of protection is defined by the appended claims rather than by the foregoing description. All changes which come within the meaning and range of equivalency of the claims are to be embraced within their scope.

Although various embodiments of the method and apparatus of the present invention have been illustrated in the accompanying Drawings and described in the foregoing Detailed Description, it will be understood that the invention is not limited to the embodiments disclosed, but is capable of numerous rearrangements, modifications and substitutions without departing from the spirit of the invention as set forth herein.

1. A system for fragmentation of adipose tissue, the system comprising:
a primary syringe;
a receiving syringe;
a mechanical processing device (MPD) with a first connector and a second connector, the first connector configured to be coupled to the primary syringe and the second connector configured to be coupled to the receiving syringe; and
a spring-loaded return device that is configured to manipulate the receiving syringe to expel fluids therefrom, wherein the return device comprises a cap with an opening configured to retain a flange of the receiving syringe, wherein the cap comprises a recessed edge configured to overlap a portion of the flange of the receiving syringe, and wherein the cap comprises a slotted notch configured to receive a fastener.

2-4. (canceled)

5. The system of claim 1, further comprising a return device coupled to the receiving syringe and configured to expel fluid from the receiving syringe.

6. The system of claim 5, wherein the return device comprises a moveable plug.

7. The system of claim 5, wherein the return device comprises a spring that biases against a plunger of the receiving syringe.

8. The system of claim 1, further comprising a syringe pump that is configured to actuate at least one of the receiving syringe or the primary syringe with a desired amount of force.

9. The system of claim 8, wherein the syringe pump is configured to provide a short burst of force to clear blockages in the connector.

10. The system of claim 1, wherein the MPD comprises:
a housing;
an adjustment control positioned at a distal end of the housing;
a conduit member positioned within the housing and configured to translate axially responsive to actuation of the adjustment control, the conduit member comprising a plurality of conduits formed therethrough,
wherein the conduit member can be positioned between the first and second connectors to form a fluid flow path therebetween that includes at least one conduit of the plurality of conduits.

11. The system of claim 10, wherein the fluid flow path allows for sterile transfer of a portion of fluids/solids to a device for particle/cell analysis.

12. The system of claim 10, wherein a conduit of the plurality of conduits comprises a textured inner surface to impart angular momentum to fluids/solids flowing therethrough.

13. The system of claim 10, wherein the MPD further comprises a chamber positioned in a flow path between the first connector and the plurality of conduits that is configured to aid the flow of fat tissue into and through the conduit.

14. The system of claim 10, wherein the housing includes a plurality of visual indicator windows that allow a user to see which conduit of the plurality of conduits is in the fluid flow path.

15. The system of claim 10, further comprising a threaded body that engages the conduit member and is configured to axially translate the conduit member.

16. The system of claim 10, further comprising a plurality of o-rings positioned between an inner wall of the housing and an outer wall of the conduit member.

17. The system of claim 16, wherein the o-rings are configured to seal off a flow path of each conduit of the plurality of conduits.

18. The system of claim 16, wherein a first conduit of the plurality of conduits includes one aperture and a second conduit of the plurality of conduits comprises two apertures.

19. The system of claim 18, wherein the diameters of the apertures of the first and second conduits are different.

20. The system of claim 1, further comprising a Biological Safety Cabinet Class II Type 2A.

21. A method of fragmenting adipose tissue, the method comprising:
transferring lipoaspirated adipose tissue to a primary syringe;
connecting a first end of a connector of the MPD to the primary syringe;
connecting a second end of a connector of the MPD to a receiving syringe;
connecting a return device to a flange of the receiving syringe;
actuating, with a syringe pump, the primary syringe to transfer the contents of the primary syringe to the receiving syringe, the actuating compressing a spring of the return device as the primary syringe is actuated; and
withdrawing the contents that were transferred to the receiving syringe to the primary syringe, the withdrawing being aided by the compressed spring,
wherein the contents are fragmented as they pass through the conduits of the MPD.

22. The method of claim 21, wherein the method is repeated for multiple diameters of the conduits of the MPD to further fragment the adipose tissue.

23. (canceled)

24. A method of enzymatic-based digestion, the method comprising:
transferring a micronized adipose tissue preparation to a first conical tube;
placing the first conical tube in a water bath to control a digestion temperature;
preparing, in a second conical tube, an enzyme digestion solution comprising collagenase, a neutral protease stock, and Dulbecco's phosphate buffered saline;
placing the second conical tube in the water bath to control the digestion temperature;
adding the enzyme digestion solution to the first conical tube;
mixing the adipose tissue preparation and the enzyme digestion solution in the first conical tube;
centrifuging the first conical tube a first time;
removing upper layers from the first conical tube to isolate a pellet in the first conical tube;
adding Dulbecco's phosphate buffered saline to the first conical tube;
centrifuging the first conical tube a second time;
removing a supernatant from the first conical tube to isolate pelleted cells in the first conical tube; and
resuspending the pelleted cells in Dulbecco's phosphate buffered saline.

25-32. (canceled)
